(19) United States
(12) Patent Application Publication
(10) Pub. No.: US 2024/0338820 A1
(43) Pub. Date: Oct. 10, 2024

(54) ARTIFICIAL INTELLIGENCE-ASSISTED GAIT ANALYSIS OF CHRISTIANSON SYNDROME PATIENTS

(71) Applicant: Brown University, Providence, RI (US)

(72) Inventors: Eric M. Morrow, Providence, RI (US); Gajendra Kumar, Providence, RI (US); Judy Shih-Hwa Liu, Providence, RI (US)

(21) Appl. No.: 18/627,737

(22) Filed: Apr. 5, 2024

Related U.S. Application Data
(60) Provisional application No. 63/457,237, filed on Apr. 5, 2023.

(51) Int. Cl. G06T 7/00; G06T 7/70; G06V 10/34; G06V 40/20; G16H 50/20

(52) U.S. Cl. CPC G06T 7/0012; G06T 7/70; G06V 10/34; G06V 40/25; G16H 50/20; G06T 2200/24; G06T 2207/10016; G06T 2207/20081; G06T 2207/30004; G06T 2207/30196

(57) ABSTRACT

Artificial intelligence-assisted gait analysis of Christianson syndrome patients is provided via recording video of a subject performing a walking task; analyzing the video to determine poses and relationships between a plurality of points of the subject corresponding to anatomical features of the subject while performing the walking task; analyzing, using a machine learning model, a gait of the subject based on the relationships between the plurality of points while performing the walking task; classifying, using the machine learning model, the subject as exhibiting or not exhibiting symptoms of a motor disorder based on the gait; and outputting, via a graphical user interface, a determination of the subject as exhibiting or not exhibiting the symptoms of the motor disorder.

### CROSS-REFERENCES TO RELATED APPLICATIONS

The present disclosure claims the benefit of U.S. Provisional Patent Application No. 63/457,237 entitled “ARTIFICIAL INTELLIGENCE-ASSISTED GAIT ANALYSIS OF CHRISTIANSON SYNDROME PATIENTS” and filed on 2023 Apr. 5, which is incorporated herein by reference in its entirety.

### BACKGROUND

Christianson syndrome (CS) is a rare X-linked neurodevelopmental disorder that affects males. The prevalence of CS is estimated to be 1:16,000 to 1:100,000. It is characterized by intellectual disability, ataxia (trouble walking due to loss of balance and movement coordination), inability to speak (absence of verbal language despite normal hearing), strabismus (crossed eyes), epilepsy, exhibit autistic features, hyperkinetic and generally happy demeanor Walking is delayed until 1-3 years of age and unsteady gait is observed due to loss of coordination of muscles leading to gait instability. These patients need continuous monitoring by parents, specialists, nurses, and caregivers. Therefore, virtual monitoring strategy is likely to provide the best care to CS patients.

Video monitoring motion capture systems are commonly used in the rehabilitation to evaluate human motor function. Human gait analysis is a quantitative approach to measure or characterize walking pattern using kinematics (joint angle).

Artificial intelligence and machine learning algorithm have been used to recognize human activity. Deep learning models can automatically extract the optimal features directly from raw spatiotemporal gait data without data preprocessing. Machine learning classifiers including k Nearest Neighbors (kNN), Light Gradient Boosting Machine (LGBM), Histogram-based Gradient Boosting (HGB), Extreme Gradient Boosting (XGB), Random Forest (RF), Support Vector Machine (SVM), Logistic Regression (LR), Decision Tree (DT), Naïve Bayes (NB), Gradient Boosting (GB), and Adaptive Boosting (AB) are commonly used. These deep learning networks have been applied to classify different Parkinson Disease states.

Accordingly, there is a need for an artificial intelligence-assisted gait analysis of Christianson syndrome patients.

### SUMMARY

Example systems, methods, and apparatus are disclosed herein for an artificial intelligence-assisted gait analysis of Christianson syndrome patients.

In light of the disclosure herein, and without limiting the scope of the invention in any way, in a first aspect of the present disclosure, which may be combined with any other aspect listed herein unless specified otherwise, a method for artificial intelligence-assisted gait analysis of Christianson syndrome patients is disclosed.

In light of the present disclosure and the above aspects, it is therefore an advantage of the present disclosure to provide users with a method for artificial intelligence-assisted gait analysis of Christianson syndrome patients.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

FIGS. 1A-1F are scatter plots that show gait patterns collected from healthy volunteers, according to embodiments of the present disclosure.

FIG. 2 is a schematic diagram depicting video acquisition, according to embodiments of the present disclosure.

FIG. 3 illustrates pose estimation and marking of body joints, according to embodiments of the present disclosure.

FIG. 4 illustrates angle estimation of hip, ankle, and ankle joints, according to embodiments of the present disclosure.

FIG. 5 is a flowchart for an example method for artificial intelligence-assisted gait analysis of Christianson syndrome patients, according to embodiments of the present disclosure.

FIG. 6 illustrates a computing device, according to embodiments of the present disclosure.

### DETAILED DESCRIPTION

Methods, systems, and apparatus are disclosed herein for a method for artificial intelligence-assisted gait analysis of Christianson syndrome (CS) patients, which may be used in the diagnosis, treatment, and (it is hoped) prophylaxis of further symptoms of the underlying syndrome. While the example methods, apparatus, and systems are disclosed herein as an artificial intelligence-assisted gait analysis of Christianson syndrome patients, it should be appreciated that the methods, apparatus, and systems may be operable with other syndromes.

Christianson syndrome (CS) is a rare X-linked neurodevelopmental disorder affecting males, characterized by symptoms including intellectual disability, inability to speak, strabismus, epilepsy, autistic features, hyperkinesis, a generally happy demeanor, and importantly, ataxia. One of the key features of CS is the progressive deterioration of gait, believed to originate from progressive cerebellar degeneration, which worsens over the first and second decades of life. This decline in motor function significantly impacts the quality of life of patients and their families; thereby, ataxia and gait represent important target for future clinical trials. Given that CS is rare and that patients are geographically distributed, there is a pressing need for effective and automated gait monitoring systems for continuous monitoring of patient remotely for future clinical trials.

Traditional methods of evaluating movements rely on subjective clinical scales assessed, in-person by trained clinicians, which can be time-consuming, prone to bias, and challenging with rare patient populations. Alternative methods, such as using computer vision and motion capture systems to measure movements, are not readily available to many clinicians or families. Hence, there is a demand for a swift, cost-effective approach capable of offering quantitative measurements of gait parameters that can be easily accessed in both home by families and in clinical settings.

Recent advances in video-based pose estimation offer a promising solution. These technologies leverage machine learning algorithms trained on diverse datasets to accurately analyze human movements from digital video inputs obtainable from omnipresent devices such as smart phones or tablets. The algorithms enable automated measurement and analysis of gait parameters with high accuracy and efficiency. This approach has several advantages. Firstly, this approach allows for convenient data collection in both home and clinical settings, eliminating the need for patients with rare disorders to visit the clinic for assessments, which may require burdensome travel, particularly for children with developmental disorders. Secondly, this approach provides quantitative measurements of gait parameters, enabling more objective evaluation of disease progression and treatment outcomes. Thirdly, this approach enables remote monitoring of patients, allowing clinicians to track changes in symptoms over time and adjust treatment strategies accordingly.

By facilitating accurate and convenient assessment of gait abnormalities, the present disclosure has the potential to advance the understanding of the natural history of CS and thereby develop outcome measures for clinical trials designed to establish more effective treatment strategy to improve the quality of life for patients and their families.

Gait analysis holds significant importance in understanding, managing, and treating gait abnormalities in CS patients. By objectively assessing and pinpointing specific gait deficits in patients while monitoring changes over time, quantitative gait analysis offers a promising pathway for treating various syndromes, such as CS. Marker-based motion capture labs are considered the gold standard, but are exorbitantly expensive and predominantly accessible only to certain hospitals and research facilities, whereas patients with rare genetic disorders such as CS are geographically distributed and may lack the means to receive care from such facilities. Other available technologies like gait mats, sensors, and wearable systems are constrained by data limitations, relatively high costs, and the necessity for specialized hardware. Hence, the present disclosure provides for artificial intelligence systems that offer a tailored gait analysis platform to quantify gait parameters for CS patients where data may be acquired in homes using commonly available devices (e.g., smartphones or tablets), thereby providing clinicians with a valuable tool for objective assessment, diagnosis, treatment planning, and monitoring. This platform enhances the understanding of gait pathology, clinical trial readiness, and improves patient outcomes through personalized interventions.

Exploratory studies have been conducted to prove the efficacy of this platform, with the aim of demonstrating the feasibility of this novel approach within a clinical trial context. An automated machine learning-enabled platform was therefore developed for quantitative human gait analysis to analyse large datasets with precision and high efficiency, provide accurate measurements of various gait parameters, offer an objective assessment of gait characteristics to eliminate potential bias introduced by human observers, ensuring consistency and reliability in the analysis results, enabling continuous real-time monitoring of gait patterns, facilitating immediate feedback and adjustment of treatment strategies. The platform was implemented via smartphones, thereby making the platform more accessible to patients in various settings, including remote or home-based environments, remotely track changes in CS patients' gait patterns and cause a treating individual to intervene when necessary. The automated and consistent evaluations offered by such a system enhance the monitoring and treatment of various movement disorders, particularly for rare disorders where patients access to clinical centers may be challenging.

In one such study, five male Christianson Syndrome (CS) patients of age ranging from 2 to 30 years, and 11 healthy volunteer controls subjects were included. These patients had been diagnosed with CS on the basis of the current diagnostic criteria and gene profiling data of International Christianson Syndrome and NHE6 Gene Network Study. Written or electronic informed and disclosure consents were obtained from legal guardians of the CS patient and volunteer subjects. Clinical information was extracted from available medical records for inclusion of patients.

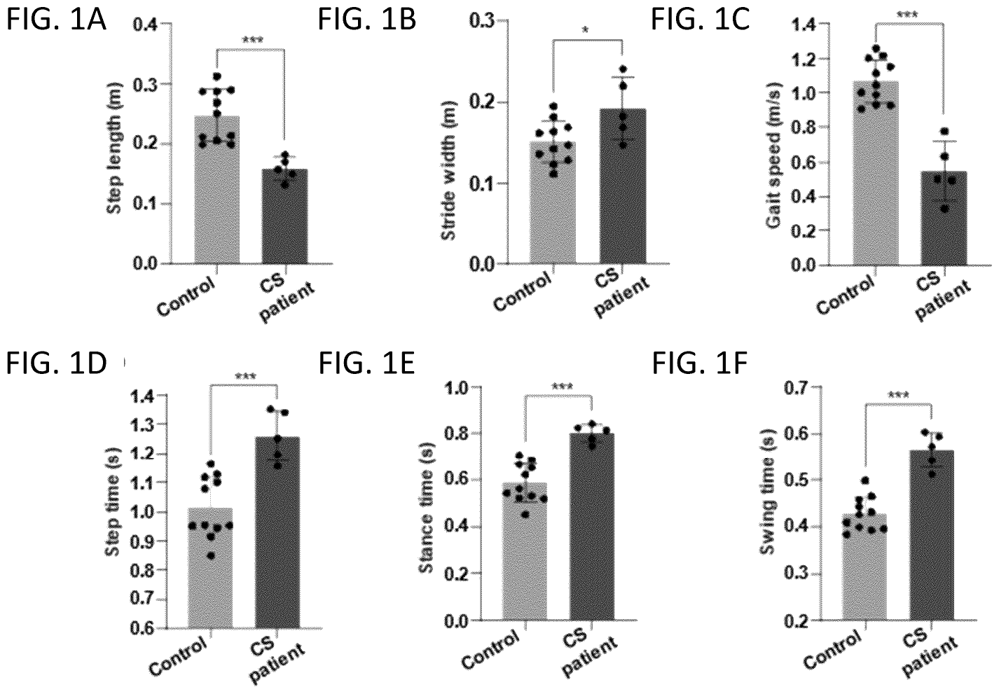

FIGS. 1A-1F are scatter plots that show gait patterns collected from healthy volunteers, according to embodiments of the present disclosure. FIG. 1A shows the swing time of the volunteers' legs, FIG. 1B shows the stance time, FIG. 1C shows the step time, FIG. 1D shows the step length, FIG. 1E shows the gait speed, and FIG. 1F shows the step width.

Table 1 provides detailed numerical values from the gait parameters illustrated in FIGS. 1A-1F along with normal ranges thereof. Data are shown as mean±standard deviation (SD, lower confidence (LC) limit at 95%, upper confidence (UC) limit at 95%, the coefficient of variation (CV), according to embodiments of the present disclosure, with data from the control/healthy subjects appearing above data from the affected subjects.

TABLE 1

| | | | | | | | % | |
|---|---|---|---|---|---|---|---|---|
| | Mean | SD | Median | LC | UC | CV | Change | Range |
| Swing | 0.427 | 0.011 | 0.425 | 0.403 | 0.451 | 8.43 | 59.96 | 0.38- |
| time | 0.565 | 0.017 | 0.573 | 0.518 | 0.612 | 6.72 | | 0.42 |
| Stance | 0.586 | 0.083 | 0.0561 | 0.530 | 0.642 | 14.21 | 27.12 | 0.59- |
| Time | 0.801 | 0.034 | 0.814 | 0.754 | 0.848 | 4.71 | | 0.67 |
| Step | 1.013 | 0.106 | 0.954 | 0.942 | 0.755 | 30.69 | 48.47 | 0.98- |
| Time | 1.260 | 0.087 | 01.253 | 1.152 | 1.085 | 10.43 | | 1.07 |
| Step | 0.248 | 0.043 | 0.251 | 0.219 | 0.276 | 17.34 | 24.38 | 0.2- |
| Length | 0.158 | 0.091 | 0.157 | 0.134 | 0.182 | 12.08 | | 0.3 |
| Gait | 1.07 | 0.126 | 1.045 | 0.981 | 1.152 | 11.86 | 36.69 | 1.0- |
| Speed | 0.547 | 0.168 | 0.502 | 0.338 | 0.755 | 30.69 | | 1.5 |
| Step | 0.151 | 0.026 | 0.147 | 0.133 | 0.168 | 17.09 | 35.21 | 0.14- |
| Width | 0.192 | 0.038 | 0.182 | 0.144 | 0.239 | 19.95 | | 0.20 |

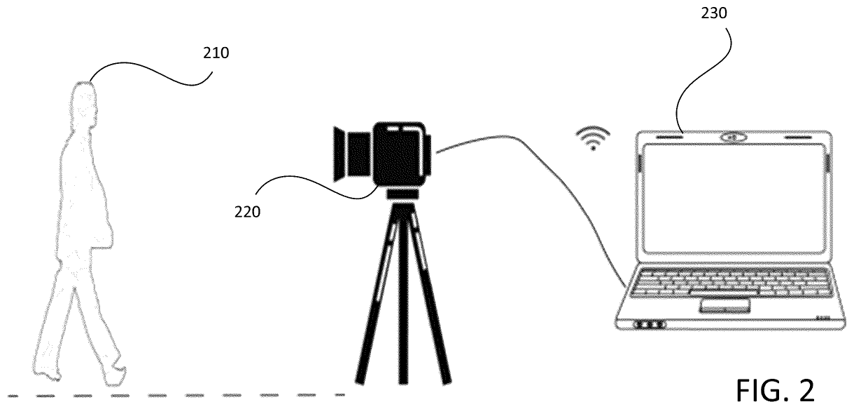

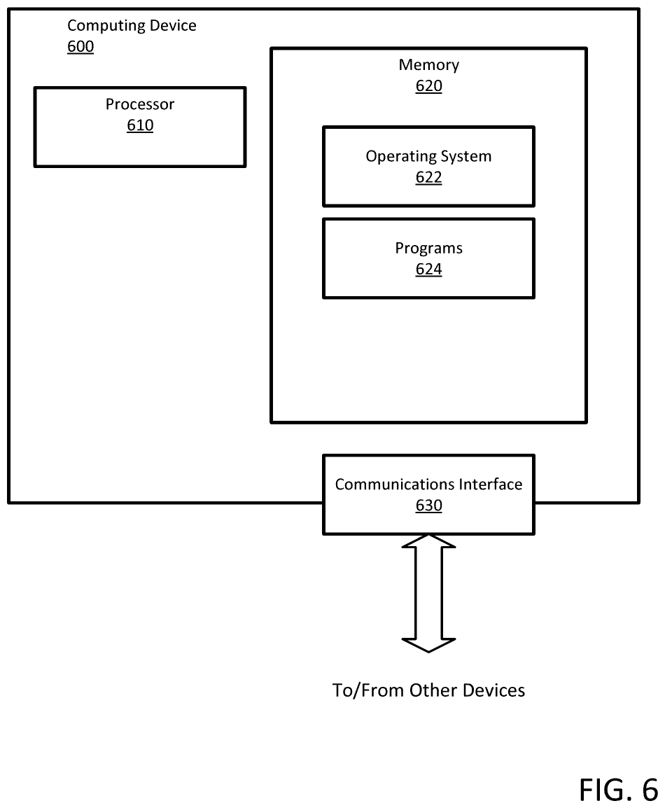

FIG. 2 is a schematic diagram depicting video acquisition, according to embodiments of the present disclosure. In various embodiments, a patient 210 is observed walking by a camera 220 connected to or associated with a computing device 230 (e.g., a computing device 600 described in relation to FIG. 6).

In some embodiments, sagittal plane (relative to the patient 210) video recordings are captured using a computing device 230 of a smartphone or tablet in which five to six steps of the patient walking are captured. In various embodiments, the computing device may discard steps from the patient initiating walking (e.g., ignoring the first X steps in a sequence captured on video) or halting walking (e.g., ignoring the last X steps in a sequence captured on video).

The camera 220, which may be integrated in or separate to the computing device 230 is placed parallel to the patient's path for walking, and at a distance to capture a given distance of walking in the field of view thereof. For example, the camera 220 may be placed at a height of approximately 75 centimetres (cm) to capture 3 meters (m) of walking path.

The patients 210 walk (or are instructed to attempt to walk) in straight line and parallel to a camera 220. Video data may be processed on the computing device 130 or downloaded from the computing device 230 for analysis.

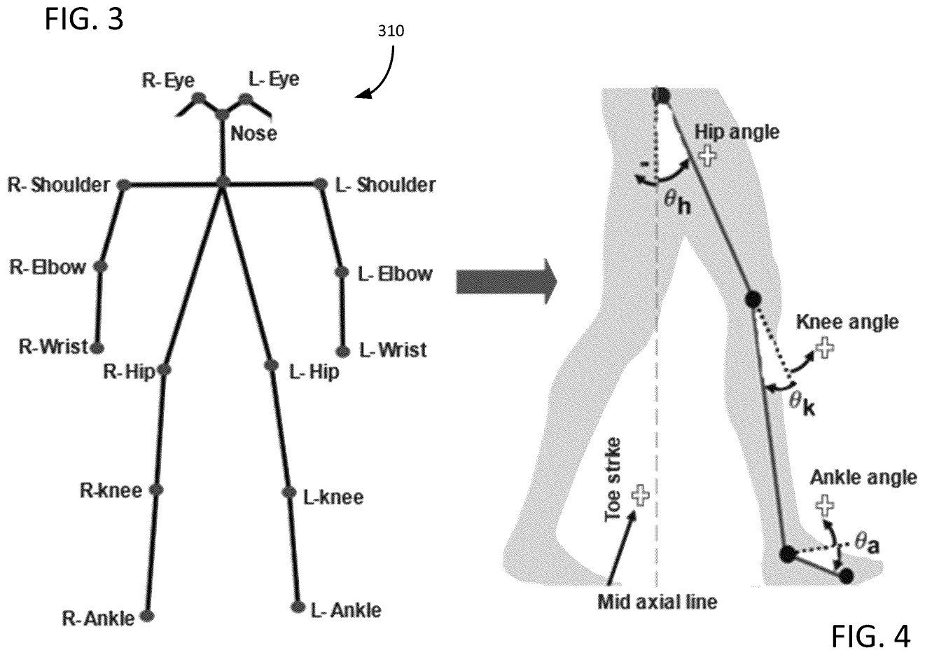

FIG. 3 illustrates pose estimation and marking of body joints, according to embodiments of the present disclosure. Using the video data captured by the camera 120 of the patient 110 walking, the system detects various key anatomical points to generate a human pose from video via sequence of images and calculate the gait kinematics of the patient. As shown in FIG. 3, thirty-two points 310 are identified, including: left eye, right eye, nose, neck, left shoulder, right shoulder, left elbow, right elbow, left wrist, right wrist, left hip, right hip, left knee, right knee, left ankle, right ankle, left heel, right heel, left hallux (big toe), right hallux, left small toes, and right small toes. As will be appreciated, not every point 310 may be identified in every image due to the position of the patient's body blocking view of some anatomical features, although the system may make estimations for where these anatomical features are located.

By tracking the locations of the marked points 310 over time, the system is able to track how the patient 220 walks. The data may be saved using various data format (e.g., in a comma-separated values (CSV) file) and shared with a model to identify gait patterns for the patients 220, which may be compared longitudinally against previous gait patterns for a given patient 220, against the gait patterns of healthy individuals, or against the gait patterns of symptomatic individuals, etc.

FIG. 4 illustrates angle estimation of hip, ankle, and ankle joints, according to embodiments of the present disclosure. A top-down or bottom-up approach may be employed to generate human pose by detecting the anatomical joint coordinates in the in the upper and lower extremities from the points 310 identified from the video The coordinate values of each joint obtained may be smoothed using a moving average filter. Next, a skeletal map with joint location was developed to define lumbar, hip, knee ankle, toe joints for left and right legs to calculate the gait kinematics (step length, step width, gait speed, step time, stance time and swing time) for the patient 220.

Gait parameters are calculated using successive heel strike and toe-off events using the number of frames as follows (1) step length=distance between successive left and right heel strikes, (2) step width=distance between two left and right heels (3) gait speed=time taken to complete one step, (4) step time=time between successive left and right heel strikes, (5) stance time=time between heel strike and toe-off of the same leg, (6) swing time=time between toe off and heel strike of the same leg. The average gait parameters for subjects were obtained by finding the mean of all these gait parameters.

Experimentally, healthy volunteers exhibited normal ranges of gait parameters such as swing time, stance time, step time, step length, gait speed, and step width, which were comparable to previously reported studies. However, CS patients demonstrated significantly decreased step length (36.29%), increased step width (27.15%), decreased gait speed (48.88%), increased step time (24.38%), stance time (36.69%), and swing time (32.32%) compared to healthy volunteers. Mean, standard deviation, median, lower and upper confidence intervals, and coefficient of variation (CV) are presented in Table 1. The highest coefficient of variation was observed in gait speed of CS patients (30.69%) compared to controls (11.86%). Using these findings, a machine learning model can be used to efficiently distinguish CS patients from healthy volunteers.

Body pose data of an individual is leveraged to track three-dimensional anatomical landmarks, which were then used for gait analysis by the machine learning model. Several sophisticated deep learning models, including k Nearest Neighbors (kNN), Light Gradient Boosting Machine (LGBM), Histogram-based Gradient Boosting (HGB), Extreme Gradient Boosting (XGB), Random Forest (RF), Support Vector Machine (SVM), Logistic Regression (LR), Decision Tree (DT), Naïve Bayes (NB), Gradient Boosting (GB), and Adaptive Boosting (AB) may be used for gait analysis to classify CS patients from healthy volunteers.

Using these data, a machine learning model is trained to identify normative data clusters and trends for various persons. Models were trained in a fully supervised manner, and various model types may be used, which offer different benefits related to individual accuracy, sensitivity, specificity and several other parameters. Model parameters were assigned randomly through the normal distribution. Following feature extraction, data were divided into training and testing sets with (in some embodiments) 80% of the data were used for training and validation and 20% were used for testing. To remove class imbalance, data of each class from the training set were resampled to equalize the number of samples for each class using the hybrid sampling Synthetic Minority Oversampling Technique with Edited Nearest Neighbors (SMOTE+ENN) analysis. The sampling method up-samples the minority data class and down-samples the majority data class, thereby improving the accuracy of the proposed model. Standardization is performed on the training and testing dataset and scaled to a mean of zero and a standard deviation of one. The resultant data were subjected into machine learning classifiers. Default parameters were used for training the ensemble models.

In various embodiments, the performance of the machine learning models is assessed by measuring the accuracy, kappa, sensitivity, specificity, F1 score, and false discovery rate (FDR). Experimentally, the kNN model exhibited highest performance with 99.02% accuracy (FIG. 2A), 99.84% receiver operating characteristic (ROC) curve, and demonstrated high predictive performance. Following kNN, the next four machine learning models, namely LGBM, HGB, XGB and RF showed more than 97% accuracy. Other machine models such as DT, GB, AB, SVM, LR, and NB also showed varying degrees of performance with more than 90% accuracy. Representative gait tracing in CS patients were distinct as compared with healthy volunteers and support the accuracy of automated gait analysis using machine learning. Accordingly, an operator may select between various model types to differentiate health and CS-affected persons.

In various embodiments, testing sets and confusion matrices are used to evaluate the models with respect to accuracy, sensitivity, specificity, F1 Score, FDR and area under ROC Curve, positive predictive value (PPV), and negative predictive value (NPV). Accuracy is described as the ratio of correct to total number of samples, with each category treated equally. Precision provide the correct proportion of the positive samples predicted by the classifier. Sensitivity is defined as the proportion of correctly predicted positive samples to all positive samples. Specificity is the proportion of correctly predicted negative samples to all negative samples. F1-score measures the weighted harmonic mean of precision and recall since precision and recall affect each other. These performance measures can be illustrated through following equations, where TP, TN, FP and FN denote true positive, true negative, false positive and false negative predictions respectively, which are obtained from confusion metrics of different ML models.

$$Accuracy = \frac{( TP + TN )}{( TP + TN + FP + FN )} \qquad Formula 1$$

$$Precision = \frac{TP}{( TP + FP )} \qquad Formula 2$$

$$Sensitivity = \frac{TP}{( TP + FN )} \qquad Formula 3$$

$$Specificity = \frac{TN}{( TN + FP )} \qquad Formula 4$$

$$f 1 - score = 2 * \frac{( Precision \times Sensitivity )}{( Precision + Sensitivity )} \qquad Formula 5$$

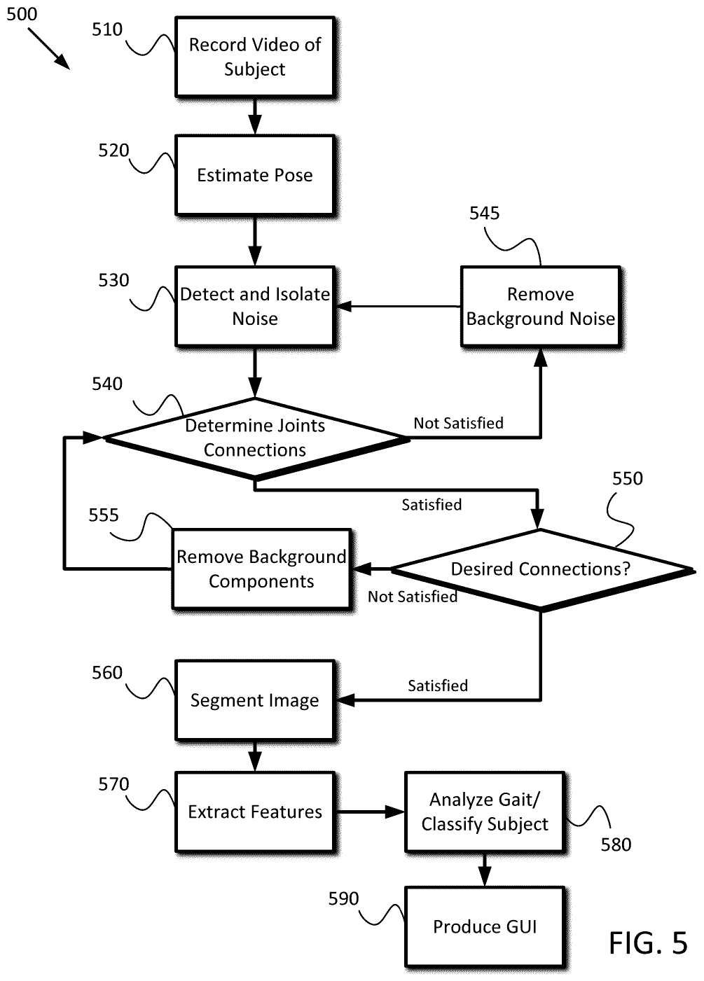

FIG. 5 is a flowchart for an example method 500 for artificial intelligence-assisted gait analysis of Christianson syndrome patients, according to embodiments of the present disclosure.

Method 500 begins at block 510, where video of a subject, who may be healthy, affected by CS or another motor disorder, or be under evaluation as healthy/affected, is recorded while performing a walking task. In various embodiments, the video is recorded while the subject is walking or attempting to walk (e.g., performing a walking task by taking at least 5-6 steps) in a straight line in a plane perpendicular to the video recording device (e.g., the sagittal plane of the subject).

At block 520, the system estimates the pose of the subject during the frames of the video collected per block 510.

At block 530, the system detects and isolates noise from the frames of the video. Various image processing techniques may be used to identify and isolate the subject from other objects in the frame so that the platform can identify the motion of the subject while performing a walking task. In various embodiments the platform generates and applies an image mask to the background or to the subject in the image to identify where the subject is located within the images of the video and isolate the subject from other persons or objects that are also included in the video.

At block 540, the platform determines the number of joints connected in the pose of the subject. When the number of joints is below a threshold number, method 500 proceeds to block 545 to further isolate the subject, whereas when the number of joints satisfies the threshold number, method 500 proceeds to block 550.

At block 545, the platform removes background noise from the still images, and returns to block 530 to reattempt to isolate the points on the subject's body while walking to analyze the gait of the subject. In various embodiments, the platform may adjust the extent of the mask or a strength at which the mask is applied to the video.

At block 550, the platform determines whether the desired number connections have been achieved. When the number of connections is below a threshold number, method 500 proceeds to block 555 to further isolate the subject, whereas when the number of connections satisfies the threshold number, method 500 proceeds to block 560.

At block 555, the platform removes small background components from the still images, and returns to block 540. In various embodiments, the small background components may include artifacts included by hair, clothing, or other objects that are shown in front of or behind the subject in one or more frames of the video, which may impede detection of the joints.

At block 560, the platform segments the images of the video into individual frames for analysis.

At block 570, the platform extracts body points corresponding to anatomical features. In various embodiments the various points on the subject's body may be identified to cross-identify other points on the subject's body and how those points move relative to one another while performing the walking task. In various embodiments, the points include one or more of the: left eye, right eye, nose, neck, left shoulder, right shoulder, left elbow, right elbow, left wrist, right wrist, left hip, right hip, left knee, right knee, left ankle, right ankle, left heel, right heel, left hallux, right hallux, left small toes, and right small toes.

At block 580, the platform analyzes the data using a machine learning model. The platform may review various gait parameters from successive motions performed as part of the walking task, which may include one or more of: a swing time; a stance time; a step time; a step length; a gait speed; and a step width. Each of the parameters may report observed maxima, observed minima, mean values, median values, standard deviations of the values, etc., which the machine learning model uses for classifying the subject as exhibiting or not exhibiting the symptoms of a motor disorder (e.g., CS). In various embodiments, this classification is based on the gait of the subject being grouped with the gaits of persons diagnosed with the motor disorder or persons not diagnosed with the motor disorder.

At block 590, the platform produces a graphical user interface (GUI) and presents the gait analysis of the subject. In various embodiments, the machine learning model is deploying as part of a web application (e.g., using Streamlit framework or other framework for deploying machine-learning models). The developed user interface app displays the result of final outcome of the machine learning analysis of the subject. The user can predict the result of the analysis from both real-time and recorded video streams. Real-time video function is achieved by incorporating a component called webRTC, which enables real-time video streaming over a network. The webRTC can be configured and customized according to the needs and requirement of the user. Gait parameters can be saved, and reports can be generated with patient details. Notably, in this method, a GUI platform presents the information to the user via visual widgets that can be manipulated without the need for command codes. In addition, the user can interact with electronic devices such as computers and smartphones through the use of icons, that respond in accordance with the programmed script, supporting user's action.

In various embodiments, as part of outputting a determination that a subject exhibits the symptoms of a motor disorder, the platform interfaces with a medical database to update electronic medical records for the subject with the determination and, additionally or alternatively one or more medical procurement systems to provide the subject with a fall alert system to help treat or manage the symptoms of the motor disorder. Additionally or alternatively, the platform interfaces with various pharmacy systems to provide the subject with various medications associated with treating or managing the motor disorder.

The present disclosure may also be understood with reference to a multi-stage approach for diagnosis and reporting to motor syndromes.

Stage 1. Gait analysis of Christianson syndrome patient using video captured motion: Video capture method is optimized first. The video capturing system consists of a smartphone (800×600-pixel images, 25-30 frames per second) mounted on top of a tripod stand at a height of 75 cm and placed parallel to the subject's walkway. The camera should be placed at a distance to capture 3 m of the walkway in its field of view. CS patients walk in straight line and parallel to a video capturing system. Video data is downloaded from the phone by the patient and sent to the user for the analysis. In alternate embodiments, the user captures the video from patient's home through wireless network.

Video processing method: The videos are processed using various algorithms (e.g., MediaPipe Pose). The user employs top-down or bottom-up approach to generate human pose by detecting the anatomical joint coordinates in the in the upper and lower extremities. Next, a skeletal map with joint location is developed. It is helpful to define the lumbar, hip, knee ankle, toc joints for left and right leg to calculate the required kinematic angles (hip, knee and ankle flexion/extension).

Gait analysis: Body parts and their coordinates in the defined frame of the video are defined. Hip joint angle (extension)=Lumbar-Hip-Knee angle-1800. Hip joint angle (flexion)=1800-Lumbar-Hip-Knee angle. Knee joint angle=1800-Hip-Knee-Ankle angle. Ankle joint angle=900-(Knee-Ankle-Toe angle)-(Ankle-Toe-Heel angle).

The coordinate values of each joint obtained by the algorithms (e.g., MediaPipe Pose) are smoothed using a five-point moving average filter. The joint angle data obtained from ten trials is analyzed and the average value will be used for final calculation.

Gait parameters are calculated using successive heel strike and toe-off events. The following the number of frames for following parameters is calculated: Step time=between successive left and right heel strikes; Stance time=between heel strike and toe-off of the same leg; Swing time=between toe off and heel strike of the same leg; Step length=distance between successive left and right heel strikes; Step width=distance between two left and right heels; and Gait speed=Time taken to complete one step.

Abnormal gait due to lower limb dysfunction leads to insufficient ankle joint dorsiflexion resulting in a foot drop. Whereas metacarpophalangeal (MP) joint flexes higher during the terminal stance phase (40-60% of the gait cycle of normal gait). Gait cycle will be marked by either a heal strike or a push-off. This method focuses on the recognition of MP joints to calculate the stance time. Gait cycle is defined as the time between two peaks of the joint angles. The average gait cycle for a subject is obtained by finding the mean of all these gait cycles. Next, the method checks the reproducibility of the data by comparing the mean and standard deviation of different observations on the same subject recorded over different sessions. Also, the method compares among different healthy subjects. Finally, CS patient's gait pattern is compared with the values from the normative database of healthy subjects. The method was tested using recorded gait video of twenty-five healthy volunteer to calculate the gait parameters. The method successfully performed the pose estimation and obtained the results. Data showed normal range of gait parameters such as swing time, stance time, step time, step length, gait speed, step width.

Stage 2. Artificial intelligence (AI) and Machine learning (ML) model for automated analysis of Christianson syndrome patient's gait: A sliding window of 5 s width that captures 3-4 full gait cycles and a fixed step length of 1 second(s) is applied to derive the gait pattern or healthy subjects and a CS patient. First, the method will test accuracy of machine learning models including k Nearest Neighbors (kNN), Light Gradient Boosting Machine (LGBM), Histogram-based Gradient Boosting (HGB), Extreme Gradient Boosting (XGB), Random Forest (RF), Support Vector Machine (SVM), Logistic Regression (LR), Decision Tree (DT), Naïve Bayes (NB), Gradient Boosting (GB), and Adaptive Boosting (AB).

Model implementation, training and performance analysis: Machine learning models are developed with TensorFlow and Pytorch under Python (Python Software) running on PC with GPU (GPU: RTX2060, 11th Gen Intel (R) Core (TM) i7-11700, 32 GB RAM). Models are trained in a fully supervised way. Model parameters are assigned randomly through the normal distribution. Following feature extraction, data is divided into training and testing sets. 80% of the data is used for training and validation and 20% is used for testing. To remove class imbalance, data of each class from the training set is resampled to equalize the number of samples for each class using the hybrid sampling Synthetic Minority Oversampling Technique with Edited Nearest Neighbors (SMOTE+ENN) analysis. The sampling method up samples the minority data class and down samples the majority data class, thereby improving the accuracy of the proposed model.

Standardization is performed on the training and testing dataset and scales them to a mean of zero and a standard deviation of one. The resultant data is subjected into machine learning classifiers Default parameters are also used for training the ensemble models of k Nearest Neighbors (kNN), Light Gradient Boosting Machine (LGBM), Histogram-based Gradient Boosting (HGB), Extreme Gradient Boosting (XGB), Random Forest (RF), Support Vector Machine (SVM), Logistic Regression (LR), Decision Tree (DT), Naïve Bayes (NB), Gradient Boosting (GB), and Adaptive Boosting (AB).

Performance of the classification models and execution of the required tasks is evaluated using metrics such as accuracy, sensitivity, specificity, precision, f1-score, kappa, and false discovery rate (FDR), area under ROC Curve, positive predictive value (PPV), and negative predictive value (NPV). Accuracy is described as the ratio of correct to total number of samples, with each category treated equally. Precision provides the correct proportion of the positive samples predicted by the classifier. Sensitivity is defined as the proportion of correctly predicted positive samples to all positive samples. Specificity is the proportion of correctly predicted negative samples to all negative samples. f1-score measures the weighted harmonic mean of precision and recall since precision and recall affect each other. As such: Accuracy=((TP+TN))/((TP+TN+FP+FN)); Precision=TP/((TP+FP)); Sensitivity=TP/((TP+FN)); Specificity=TN/((TN+FP)); and f1-score=2*((Precision×Sensitivity))/((Precision+Sensitivity)); where TP, TN, FP and FN denote true positive, true negative, false positive and false negative predictions respectively.

Stage 3. Development of graphical user interface (GUI) with AI and ML integrated platform for real-time gait detection of Christianson syndrome patient: the method integrates the developed artificial intelligence (AI) model of highest accuracy, sensitivity, specificity, precision and f1-score with GUI by deploying it into a web application using Streamlit framework. Streamlit is a free open-source framework for deploying machine-learning models. Streamlit is used due to its advantages such as smooth, interactive web app building with python without domain expertise in web development. The developed user interface app displays the result of final outcome. The user can predict the result from both real-time and recorded video streams. Real-time video function is achieved by incorporating a component called webRTC, which enables real-time video streaming over a network. The webRTC can be configured and customized according to the needs and requirement of the user. Gait parameters can be saved, and reports can be generated with patient details. Notably, in this method a graphical user interface (GUI) platform presents the information to the user via visual widgets that can be manipulated without the need for command codes. In addition, the user can interact with electronic devices such as computers and smartphones through the use of icons, that respond in accordance with the programmed script, supporting user's action.

FIG. 6 illustrates a computing device 600, as may be used for artificial intelligence-assisted gait analysis of Christianson syndrome patients, according to embodiments of the present disclosure. The computing device 600 may include at least one processor 610, a memory 620, and a communication interface 630.

The processor 610 may be any processing unit capable of performing the operations and procedures described in the present disclosure. In various embodiments, the processor 610 can represent a single processor, multiple processors, a processor with multiple cores, and combinations thereof.

The memory 620 is an apparatus that may be either volatile or non-volatile memory and may include RAM, flash, cache, disk drives, and other computer readable memory storage devices. Although shown as a single entity, the memory 620 may be divided into different memory storage elements such as RAM and one or more hard disk drives. As used herein, the memory 620 is an example of a device that includes computer-readable storage media, and is not to be interpreted as transmission media or signals per se.

As shown, the memory 620 includes various instructions that are executable by the processor 610 to provide an operating system 622 to manage various features of the computing device 600 and one or more programs 624 to provide various functionalities to users of the computing device 600, which include one or more of the features and functionalities described in the present disclosure. One of ordinary skill in the relevant art will recognize that different approaches can be taken in selecting or designing a program 624 to perform the operations described herein, including choice of programming language, the operating system 622 used by the computing device 600, and the architecture of the processor 610 and memory 620. Accordingly, the person of ordinary skill in the relevant art will be able to select or design an appropriate program 624 based on the details provided in the present disclosure.

The communication interface 630 facilitates communications between the computing device 600 and other devices, which may also be computing devices as described in relation to FIG. 6. In various embodiments, the communication interface 630 includes antennas for wireless communications and various wired communication ports. The computing device 600 may also include or be in communication, via the communication interface 630, one or more input devices (e.g., a keyboard, mouse, pen, touch input device, etc.) and one or more output devices (e.g., a display, speakers, a printer, etc.).

Although not explicitly shown in FIG. 6, it should be recognized that the computing device 600 may be connected to one or more public and/or private networks via appropriate network connections via the communication interface 630. It will also be recognized that software instructions may also be loaded into a non-transitory computer readable medium, such as the memory 620, from an appropriate storage medium or via wired or wireless means.

Accordingly, the computing device 600 is an example of a system that includes a processor 610 and a memory 620 that includes instructions that (when executed by the processor 610) perform various embodiments of the present disclosure. Similarly, the memory 620 is an apparatus that includes instructions that, when executed by a processor 610, perform various embodiments of the present disclosure.

Certain terms are used throughout the description and claims to refer to particular features or components. As one skilled in the art will appreciate, different persons may refer to the same feature or component by different names. This document does not intend to distinguish between components or features that differ in name but not function.

As used herein, the term “optimize” and variations thereof, is used in a sense understood by data scientists to refer to actions taken for continual improvement of a system relative to a goal. An optimized value will be understood to represent “near-best” value for a given reward framework, which may oscillate around a local maximum or a global maximum for a “best” value or set of values, which may change as the goal changes or as input conditions change. Accordingly, an optimal solution for a first goal at a given time may be suboptimal for a second goal at that time or suboptimal for the first goal at a later time.

Various units of measure may be used herein, which are referred to by associated short forms as set by the International System of Units (SI), which one of ordinary skill in the relevant art will be familiar with.

As used herein, various terms provided with reference to the body of a biological subject are to be understood with reference to the standard anatomical position of that biological subject using anatomical terms of location e.g., as set by the International Federation of Associations of Anatomists or the World Associate of Veterinary Anatomists that will be understood by the person on ordinary skill in the relevant art without further explanation.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112 (f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

We claim:

1. A method, comprising:
recording video of a subject performing a walking task;
analyzing the video to determine poses and relationships between a plurality of points of the subject corresponding to anatomical features of the subject while performing the walking task;
analyzing, using a machine learning model, a gait of the subject based on the relationships between the plurality of points while performing the walking task;
classifying, using the machine learning model, the subject as exhibiting or not exhibiting symptoms of a motor disorder based on the gait; and
outputting, via a graphical user interface, a determination of the subject as exhibiting or not exhibiting the symptoms of the motor disorder.

2. The method of claim 1, wherein the relationships between the plurality of points include gait parameters including one or more of:
a swing time;
a stance time;
a step time;
a step length;
a gait speed; and
a step width.

3. The method of claim 1, wherein the plurality of points include: left eye, right eye, nose, neck, left shoulder, right shoulder, left elbow, right elbow, left wrist, right wrist, left hip, right hip, left knee, right knee, left ankle, right ankle, left heel, right heel, left hallux, right hallux, left small toes, and right small toes.

4. The method of claim 1, further comprising:
supplying the subject with a fall alert system in response to determining that the subject exhibits the symptoms of the motor disorder.

5. The method of claim 1, wherein the video is taken in a sagittal plane of the subject.

6. The method of claim 1, wherein analyzing the gait further comprises:
noise smoothing and removal of background objects from the video.

7. The method of claim 1, wherein the machine learning model is trained as a k Nearest Neighbors deep learning model.

8. A system, comprising:
a processor; and
a memory, including instructions that when executed by the processor perform operations that include:
recording video of a subject performing a walking task;
analyzing the video to determine poses and relationships between a plurality of points of the subject corresponding to anatomical features of the subject while performing the walking task;
analyzing, using a machine learning model, a gait of the subject based on the relationships between the plurality of points while performing the walking task;
classifying, using the machine learning model, the subject as exhibiting or not exhibiting symptoms of a motor disorder based on the gait; and
outputting, via a graphical user interface, a determination of the subject as exhibiting or not exhibiting the symptoms of the motor disorder.

9. The system of claim 8, wherein the relationships between the plurality of points include gait parameters including one or more of:
a swing time;
a stance time;
a step time;
a step length;
a gait speed; and
a step width.

10. The system of claim 8, wherein the plurality of points include: left eye, right eye, nose, neck, left shoulder, right shoulder, left elbow, right elbow, left wrist, right wrist, left hip, right hip, left knee, right knee, left ankle, right ankle, left heel, right heel, left hallux, right hallux, left small toes, and right small toes.

11. The system of claim 8, wherein the operations further comprise:
supplying the subject with a fall alert system in response to determining that the subject exhibits the symptoms of the motor disorder.

12. The system of claim 8, wherein the video is taken in a sagittal plane of the subject.

13. The system of claim 8, wherein analyzing the gait further comprises:
noise smoothing and removal of background objects from the video.

14. The system of claim 8, wherein the machine learning model is trained as a k Nearest Neighbors deep learning model.

15. A non-transitory computer readable storage medium, including instructions that when executed by a processor perform operations comprising:
recording video of a subject performing a walking task;
analyzing the video to determine poses and relationships between a plurality of points of the subject corresponding to anatomical features of the subject while performing the walking task;
analyzing, using a machine learning model, a gait of the subject based on the relationships between the plurality of points while performing the walking task;
classifying, using the machine learning model, the subject as exhibiting or not exhibiting symptoms of a motor disorder based on the gait; and
outputting, via a graphical user interface, a determination of the subject as exhibiting or not exhibiting the symptoms of the motor disorder.

16. The storage medium of claim 15, wherein the relationships between the plurality of points include gait parameters including one or more of:
a swing time;
a stance time;
a step time;
a step length;
a gait speed; and
a step width.

17. The storage medium of claim 15, wherein the plurality of points include: left eye, right eye, nose, neck, left shoulder, right shoulder, left elbow, right elbow, left wrist, right wrist, left hip, right hip, left knee, right knee, left ankle, right ankle, left heel, right heel, left hallux, right hallux, left small toes, and right small toes.

18. The storage medium of claim 15, wherein the operations further comprise:
supplying the subject with a fall alert system in response to determining that the subject exhibits the symptoms of the motor disorder.

19. The storage medium of claim 15, wherein the video is taken in a sagittal plane of the subject.

20. The storage medium of claim 15, wherein analyzing the gait further comprises:
noise smoothing and removal of background objects from the video.
